(19) United States
(12) Patent Application Publication
(10) Pub. No.: US 2007/0068524 A1
(43) Pub. Date: Mar. 29, 2007

(54) Inhaler for moisture sensitive drugs

(75) Inventors: Thomas Nilsson, Mariefred (SE); Lars Olov Emanuel Holwaster, Straengnaes (SE)

Correspondence Address: OBLON, SPIVAK, MCCLELLAND, MAIER & NEUSTADT, P.C., 1940 DUKE STREET, ALEXANDRIA, VA, 22314, US

(73) Assignee: Microdrug AG, Hergiswil (CH)

(21) Appl. No.: 11/255,011

(22) Filed: Oct. 21, 2005

(30) Foreign Application Priority Data
Sep. 28, 2005 (SE) 0502146-4

(51) Int. Cl. A61M 15/00; A61M 16/10

(52) U.S. Cl. 128/203.15; 128/203.12

(57) ABSTRACT

A dry powder inhaler device (DPI) is disclosed. When a user activates the inhaler, the DPI is capable of delivering a dry powder dose directly from a medicament container, loaded into the DPI. A method is also disclosed for delivering a dry powder medicament dose directly from a container to a user of a DPI, whereby a sealing foil of the container is being slit open concurrently with aerosolizing and entraining of the powder in the dose into the inhaled air.

### REFERENCE TO PRIOR APPLICATIONS

This application claims priority to Swedish patent application SE0502146-4 filed Sep. 28, 2005, incorporated herein by reference.

### TECHNICAL FIELD

The present invention relates to a dry powder inhaler device (DPI) for metered dry powder medicament doses, and particularly to a DPI capable of delivering moisture sensitive drugs in humid ambient conditions with only a small drop in performance compared to normal ambient conditions.

### BACKGROUND

The dosing of drugs is carried out in a number of different ways in the medical service today. Within health care there is a rapidly growing interest in the possibility of administering medication drugs as a powder directly to the airways and lungs of a patient by means of an inhaler in order to obtain an effective, quick and user-friendly delivery of such substances. The active substance in dry powder form, suitable for inhalation needs to be finely divided so that the majority by mass of particles in the powder is between 1 and 5μm in aerodynamic diameter (AD). Powder particles larger than 5μm tend not to deposit in the lung, when inhaled, but to stick in the mouth and upper airways where they are medicinally wasted and may even cause adverse side effects.

In WO 02/00280 A2 and U.S. Pat. No. 6,655,381 B2, an inhaler comprising a magazine holding a rigid unitary magazine including a plurality of integral reservoirs is described. Each reservoir will hold a pre-metered dose of dry powder sealed with a foil.

In WO 03/66470 A1, GB 02 385 020 A, and WO 03/15857 A1 an inhaler using compartments to hold the pharmaceutical formulation is described. The compartments have a first and a second face that will be sealed with a foil. A separate part inside each compartment is designed to rupture the foil before inhalation and the documents discuss weakening special sections in the foil to make the opening easier and more reliable.

In WO 01/30430 A1 a dosage unit for dry powder medicaments is described. The dosage unit is possible to incorporate into a dry powder inhaler such as the one described in WO 02/00279, the dosage unit having a slidable chamber in a sleeve and an openable closure member possible to fit into the dry powder inhaler device. The dosage unit is described to have a cover of substantially the same diameter as the sleeve or being of a frangible material. A separate part inside the device will then push the cover open or rupture the frangible material.

In US 2002/0033176 A1 a dry powder medicament inhalator is described, which is possible to load with a medicament cartridge. The inhalator uses an inhalation activated flow-diverting means for triggering the delivery of the medicament using a lancet to penetrate the medicament cartridge.

Dose inhalers of prior art, as in the above examples, often leave the powder dose exposed to the surrounding atmosphere for a long time before the dose is actually delivered. This is due to the inhaler design and the design of the dose container. Barrier properties of the container embodiments are also an issue. Adequate protection must be secured of the fine particle dose of the enclosed medicament during transportation, storing and in-use. Some prior art products make it necessary to open the container and empty the dose into an aerosolizing chamber before the user can begin an inhalation cycle. In some cases the dose may get exposed to a voluntary or involuntary exhalation from the user before a proper inhalation cycle begins. In some inhalers the container is opened by a first action by the user but the act of inhaling from the opened container is delayed uncontrollably, because the user is somehow distracted. Exposing the powder dose to the atmosphere for any reason, including technical shortcomings of the container-inhaler combination, must be kept as short as possible so that the quality of the dose cannot deteriorate before it is inhaled.

Because inhalable drugs are attracting a lot of interest today, many new formulations of old and new medicaments are now in development into inhalable dry powders. The objective is to present dry, inhalable powder formulations and have them approved for treatment of local or systemic disorders by means of inhalation to the airways and lungs. However, quite a few of these formulations are very sensitive to humidity. Thus, new demands arise on dry powder inhalers and their ability to maintain acceptable performance in terms of delivered dose mass, dose uniformity and fine particle fraction of the delivered dose when ambient conditions change from the ideal ones, e.g. when administering doses in very humid conditions.

For various reasons many such new dry powder drugs are sensitive to exposure to moisture, not only long term but also extremely short-term exposure. Inhaling these new drugs using a prior art DPI may provide acceptable dosage performance in normal, dry, ambient conditions, but the dosage performance from the DPI drops dramatically if the inhalation is performed in ambient air of high humidity. This is often the case even if the dose is well protected up to the point of administration by the DPI. The DPI and the method of aerosolizing the powder dose play a big role in this problem. If the drug delivery performance varies depending on ambient conditions, the medical efficacy of the drug will vary uncontrollably too much.

Thus, there is a need for improved dry powder inhalers guaranteeing consistent high quality administration of dry powder doses under varying ambient conditions.

### SUMMARY OF THE INVENTION

The present invention presents a novel method of boosting the dose delivery performance of dry powder inhaler devices in humid ambient conditions, such that the inhaler performance is kept within tight limits all the way from ideal to very hot and humid ambient conditions. The invention discloses a novel use in the pharmaceutical industry of well-known desiccant materials normally used to keep medical products dry during transportation and storage.

The present invention teaches that a dynamic drying of humid air, which is drawn into an inhaler device for releasing a dry powder dose, reduces the drop in performance connected to humidity in the air releasing the dose. The invention is particularly useful in connection with many dry powder drug formulations, now being introduced or in development, which are sensitive to high relative humidity in the air. Doses will, for example, form particle agglomerates very quickly, even when exposed for a very short period to humidity, like in an inhaler in preparation before the dose is actually sucked up by a user of the device. Such agglomerates cannot be broken up into free particles by the inhaler device upon inhalation. The present invention, however, prevents these agglomerates from forming.

In one preferred embodiment of the invention a desiccant material, according to the invention, is placed in an air channel of an inhaler device between an air inlet and a dose prepared for release by a stream of air. A flow of air passes through the desiccant material before reaching the dose, whereby the desiccant adsorbs some or all of the humidity in the air. The relative humidity of the air after having passed through the desiccant is thus less, preferably significantly less, than the relative humidity before the desiccant. By taking such an approach, the reduction in relative humidity of the air applied to the powder dose in order to release and entrain it into the air has surprisingly proven to extend the performance enormously of the inhaler device for sensitive drugs.

In still another aspect of the present invention a dry powder inhaler device is disclosed, which is adapted to receiving a dose container with an enclosed metered dose. Preferably, an opener opens the container when at least a minimum suction has been applied to the device, not before, and the powder of the enclosed dose in the container is released into air and sucked up by a user of the inhaler device.

### BRIEF DESCRIPTION OF THE DRAWINGS

The invention, together with further objects and advantages thereof, may best be understood by referring to the following detailed description taken together with the accompanying drawings, in which:

FIG. 1 illustrates a diagram of relative humidity and temperature for air having passed through a desiccant

FIG. 2 illustrates a side view of a preferred embodiment of an inhaler device, and

FIG. 3 illustrates a side view of another preferred embodiment of an inhaler device.

### DESCRIPTION OF THE INVENTION

The present invention discloses a novel type of dry powder inhaler device (DPI), which is suitable for all types of dry powder drug formulations, but particularly advantageous for moisture sensitive dry powders. By introducing a desiccant material into one or more of the air channels of the inhaler device, we have surprisingly found that it is possible to reduce the relative humidity of the flowing air before the air-stream reaches the dry powder dose. The dose may be pre-metered and introduced into the device e.g. in a tight blister or capsule, which is opened just before delivery to an inhaling user. Alternatively, the dose may also be metered from a bulk store inside the inhaler device prior to delivery by inhalation. Regardless of what type of DPI is preferred, the novel use of desiccant in the upstream air channels of a DPI, according to the present invention, provides a major improvement in the drug delivery performance of the inhaler device, particularly in high humidity conditions. Surprisingly, the disclosed invention can be put to use in most DPI types and it can be used to boost the performance of well-proven inhaler devices and make them into inhaler devices for moisture sensitive drug formulations, the inhalation of which are a problem that this invention solves.

A use of desiccants in inhalers is known in the art, but the use is intended for and arranged for keeping internal parts dry, especially if powder is available from a bulk source. Desiccants are kept out of the air channels in prior art devices in order not to create loss of air pressure during inhalation, which is crucial to the drug delivery efficacy of most prior art devices. Another reason the prior art keeps the desiccant out of the air channels is to save the desiccant from being consumed. Persons, skilled in the art, have never before seen a benefit from using desiccants in the novel manner presented in the present invention.

We have surprisingly found that in a particular embodiment of the invention it is possible to fill, at least partly, the internal air channels of an inhaler device with enough desiccant material to last the specified in-use period of the inhaler device, even if the device is used in harsh and very humid conditions. In many parts of the world, people and potential users find themselves in hot and humid climates, where ambient humidity may be as high as 75% Rh or higher. Of course, a safe treatment of human disorders based on inhaled medicaments must provide stable, predictable drug delivery to all users in foreseeable situations of usage, including humid ambient conditions. In-process drying of the flowing inhalation air every time the user inhales a dose of a dry powder drug is an effective method, we find, of improving and securing the level of performance from a dry powder inhaler, i.e. performance in terms of high, stable, medical efficacy of the drug, even in very humid conditions of use.

In a further embodiment of the invention, the inhaler device is provided with means to open and close the air inlet of the device such that the inlet opens to let inhalation air enter the device when the dose is about to be administered. A suction effort made by a user generates airflow into an air channel of the device, such that the air is first directed towards the desiccant. The flowing air passes, at least partly, through the desiccant material, where moisture in the air is adsorbed and/or absorbed by the desiccant before the dried air-stream hits the dose container or aerosolisation chamber where the dose to be sucked up is located. Fortunately, it is not necessary to remove all moisture from the incoming air, because most dry powder drugs do not deteriorate chemically or physically in a linear proportion to relative humidity. Generally, dry powders show a nonlinear, typically exponential deterioration rate with increasing relative humidity. Thus, it is only meaningful to remove the excess moisture over a certain Rh-value, whereby the relative humidity of the air having passed through the desiccant is reduced below a certain, safe threshold value of relative humidity as % Rh, such that short-term deterioration of the powder dose by the remaining moisture in the air-stream is prevented.

Short-term deterioration of dry, medicament powders is generally not determined by chemical degradation, but is rather more physical in nature. Humidity in the surrounding air may be very quickly adsorbed by the powder particles. Depending on the degree of hydrophobicity or hydrophilicity of the powder this process of adsorption of moisture from the air is more or less rapid and more or less pronounced. Some powder formulations tend to act as drying agents, i.e. gaining weight extremely fast by water adsorption or absorption in humid air. Thus, it is important to study the water sorption isotherm for a powder formulation before deciding which DPI is best suited to use for administration of doses thereof. For instance, water on the surface of small, inhalable particles may be harmless up to a point where the number of water droplets on a particle have increased so that the droplets connect to water droplets on neighboring particles, whereby particle agglomerates form that are held together by strong inter-particle forces. Such agglomerates are very difficult to de-aggregate by the DPI. Keeping the humidity in the air below a critical point for the particular medicament powder is thus important, as soon as the dose is being exposed to air before the dose is released and entrained into inhalation air. What relative humidity in air is critical to drug delivery performance depends largely on the powder and the formulation, but typically the threshold value, not to be exceeded, is in a range from 40 to 70% Rh.

According to the invention it is normally not necessary to dry the inhalation air beyond a certain point, as discussed above, for the combination of a selected inhaler and the powder dose, which is going to be inhaled. Thus, a desiccant should be selected which adsorbs water from air predominantly at and above the critical relative humidity, i.e. x % Rh, where x is typically any number between e.g. 40 and 80. The selected desiccant material is preferably much less active below this threshold x. A successful selection of an ideal desiccant in this respect means that less desiccant can be used in the inhaler compared to a different desiccant, which also adsorbs water at lower relative humidity. Such a desiccant will be saturated before the ideal one, given the same number of doses and ambient conditions, thereby requiring more desiccant to compensate for the tendency to adsorb more water than strictly necessary. Furthermore, the less desiccant mass that is used means less pressure loss over the desiccant, which in turn means that more suction power is available for the job of releasing and de-aggregating the powder in the dose. Typically, pressure loss across the desiccant may be 2-20% of the applied suction pressure during inhalation, the particular application sets what may be an acceptable value. The resulting air speed through the desiccant should be low, preferably not higher than 2-3 m/s, more preferably below 1 m/s to allow the air enough duration of stay for the desiccant to adsorb as much water molecules from the air as possible within a specification framework. One of ordinary skill is able to select such desiccants given the present disclosure including the non-limiting listing of desiccants below.

In a further aspect of the invention, active inhalers including the ones using so called spacers also benefit from the disclosure. Active inhaler devices often use pressurized gas, e.g. ambient air to aerosolize the dose before it is inhaled. Some devices use a spacer, i.e. a large receiver, into which the aerosolized dose is taken as a dust cloud. Normally, a user pumps up the pressure in a reservoir chamber or pressurized gas from a canister is used instead, prior to an inhalation. The pressurized air is then let out through an outlet inside the device, such as a valve, and the air is directed onto the dose with high air speed, which releases the dose and the aerosolized dose is then inhaled either directly or indirectly through a spacer arrangement. Advantageously, the desiccant in this case is arranged at the air inlet, such that the ambient air being pumped into the reservoir chamber is first dried by the desiccant, at least partly. The pressurized air in the chamber will be reduced in relative humidity when let out onto the dose, which improves the performance of the inhaler device regarding sensitive drugs.

Desiccants suitable for use in the present application are typically but non-exclusively silica gels (SiO₂), activated alumina (Al₂O₃), molecular sieves and clays. Each material has advantages and disadvantages. For example, silica gels generally have a quick response time, which is very suitable for dynamic applications, typical of inhaler applications as described in the foregoing. Silica gels also have a high adsorption capacity (saturation approximately at 35% weight increase) and high efficacy in relative humidity between 40 and 80% Rh, which is perhaps the most interesting humidity range for inhaler applications. Activated alumina, on the other hand, have a higher adsorption capacity (saturation approximately at 42% weight increase) but are slower in the response to dynamic conditions. Molecular sieves have less adsorption capacity than aforementioned types, but they are generally very good at adsorption in low relative humidity, e.g. below 40% Rh. Of course, in any particular application for the present invention, it may be desirable to combine different desiccants in order to combine the best qualities from different types or from differently acting desiccants of the same type to meet the requirements in the particular case.

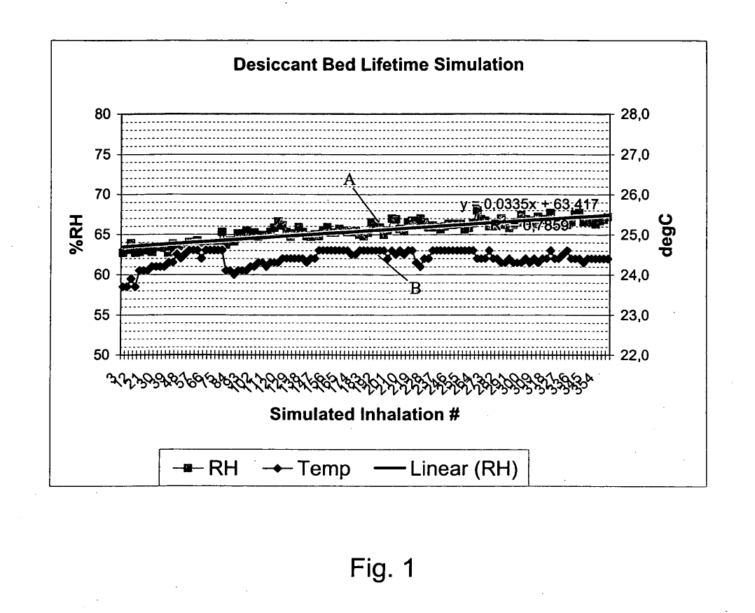

FIG. 1 illustrates a test of the invention in a diagram showing how temperature (curve B) and relative humidity (curve A) of the inhaled air after a silica gel desiccant varies over a long time and several hundred of simulated inhalations, when the inhaler device is used in ambient conditions of 25 C/75% Rh. As can be seen the inhaled air is much reduced in humidity over the whole test period.

In a non-limiting, illustrative embodiment of the present invention a silica gel is used for adsorbing humidity in excess of 65% Rh. The amount of silica gel is selected with regard to the size and volume of air channel in the inhaler device, the number of doses of a selected medicament formulation that the device is specified to deliver, which typically is between 100 and 500 off. Typically, the amount of gel necessary to provide safe and consistent drug delivery performance for the full in-use time of the device is in a range from 2 to 20 g dry mass. Preferably, a type of gel is selected, which comprises dust-free, spherical, biologically acceptable particles, which do not change in size or disintegrate when saturated. Crushed gel particles, common in the industry, should be avoided, because they have a wide range of particle sizes and present much more of a problem from a regulatory aspect, because of the potential risk of emitting dust particles into the inhalation air. Of course, in any embodiment of the invention, i.e. having desiccants in the air channels of an inhaler device, adequate filter protection or the like may be necessary to incorporate in order to eliminate the risk of inhaling unwanted dust particles. Another possibility is to use dust-free drying agents of suitable particle size for the application as desiccant.

In yet a non-limiting, illustrative embodiment of the present invention a so-called monolith extruded from e.g. silica gel, clay or zeolite is used, said monolith presenting a honeycomb structure, similar to an automotive catalytic converter, e.g. formed to physically suit a space in the air channel of a selected inhaler device. The honeycomb structure makes the active surface extremely large per weight of the material used, which may be advantageously used in the inhaler device application.

In a further aspect of the invention, the inhaler device is closed when not in use, such that ambient air is prevented, as far as possible, to enter the device. The desiccant is thereby preserved and not consumed unnecessarily. The desiccant is predominantly in use only during inhalation of doses. The inhaler device is therefore preferably provided with means to close the air inlet, such as a flap or valve, behind which the desiccant is located. If all exterior ports of the device are closed after use, the internal surfaces and the internal air volume will be dried out while the device is closed. Later, when the device is opened in preparation for use shortly before delivery of a next dose, the initial air being inhaled by the user is dry, which is a further advantage of the present invention.

One embodiment of the invention is very suitable for inhaler devices using blisters or capsules containing a metered dose, where the dose container is first opened inside the device in an opening operation to be followed in a next step by an act of inhalation. It is common in these devices that a suction-induced flow of air follows after opening of the dose container. Since the internal inhaler space in this case is preferably filled with dry air just prior to use, according to the present invention, the effect of an interval of dose exposure to the internal air of the device is negligible.

In a preferred embodiment of the invention, the inhaler device is provided with alternative routes for the inhaled air. The objective is here to let the air-stream pass through the desiccant, but only while the dose is in the process of being released and entrained into the air-stream and delivered to an inhaling user. Typically, release and delivery of a medicament dose by inhalation takes less than a second, the exact release process and the timing of it depends mainly on the design of the inhaler device, the dose container and how the dose is made available for inhalation. However, a suction effort resulting in a deep inhalation, which is normally recommended for drug delivery by inhalation, lasts for up to 5 seconds or more. Thus, several seconds of inhalation are preferably used to push the dose into the lung and to let the particles of the dose sediment onto the mucous membrane. It is normally not necessary to let the air following on the release of the dose pass through the desiccant whereby the air would be dried, since the dose, after its release, is already in the airways of the user.

Advantageously, the inhaler device is provided with means not only to close the air inlet to the desiccant when the inhalation is over, but also to comprise means by which the flow of air is diverted as soon as the dose has been released, such that the airflow bypasses the desiccant while the inhalation is brought to an end.

In yet a further aspect of the invention, the desiccant is filled in a cartridge adapted for insertion into a corresponding air channel in a selected inhaler device. The cartridge may then be removed and discarded and a new one inserted either at regular intervals or the cartridge may be regenerated by the user of the inhaler device and used again, e.g. if the inhaler is intended for a long life of administering a large number of doses before scrapping. The cartridge may signal by color change, for instance, or by a dose counter or other signaling means when the cartridge is due to be exchanged or regenerated by the user.

In a different embodiment of the invention, such as in the case of an inhaler administering pre-metered single or combined doses of medicaments from dose containers, such as blisters or capsules, desiccant material is integrated in the container as either an added component or integrated in the container material as such. The airflow is forced by the internal air channels of the inhaler device to pass through the desiccant, before the air reaches into the dose container to release the dose, whereby the air is dried before releasing the dose. In this case it is possible to exclude all or part of the desiccant otherwise necessary to be incorporated into the device itself.

In a preferred embodiment of the present invention the user pushes a slide carrying the dose in a sealed container into the inhaler body during an interval of between 0.1 and 5 s, although preferably between 0.2 and 2 s. The slide is thus manually pushed with a generally constant speed using a relatively light force at the same time as he or she inhales through a mouthpiece of the inhaler. The motion of the slide brings the container seal into contact with an opener inside the inhaler. The opener opens the foil and folds it away from the enclosed dose. This action makes the dose available to a suction nozzle, such that the stream of air entering the inhaler flows through the desiccant material of the present invention, at least partly, and then into the inlet aperture of the suction nozzle at high speed at this point. The dose is thereby released, aerosolized and de-aggregated gradually while the dose container is being carried past the foil opener at the same time as the dose is carried past the suction nozzle by the user operated slide.

Preferably, the slide is locked by a catch in its first, container loading position so that the slide cannot move when the user exerts force on the slide. The catch lets go of the slide when the user also applies a certain minimum suction effort to the mouthpiece of the inhaler. Then, a flap or similar arrangement known in the art opens for air to be sucked in through the desiccant. The user can now push the slide and dose container into the inhaler body while inhaling, whereby the dose gets delivered gradually. Optionally, the flap itself, or additional means for controlling opening and closing of an air inlet, lets air pass through the desiccant during the motion of the slide, but closes the air inlet to the desiccant material once the slide is brought fully into the inhaler body. At that point the inlet airflow is diverted to bypass the desiccant for the remaining interval of the inhalation effort. This optional embodiment reduces the consumption of the desiccant material, thereby extending the useful lifetime of the desiccant.

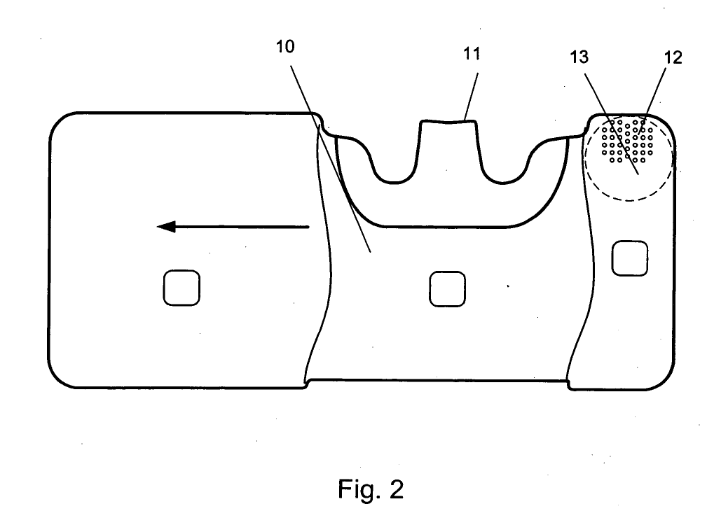

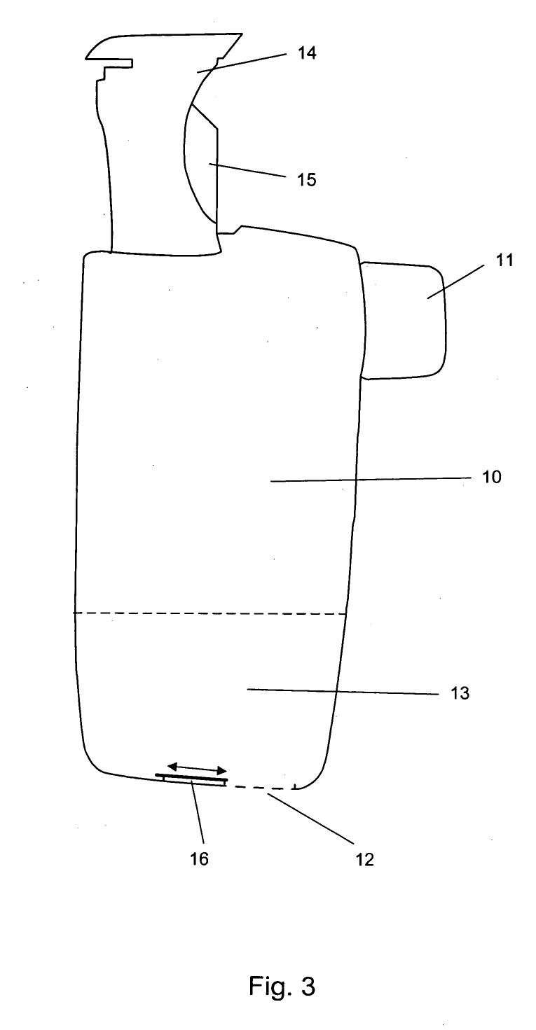

In FIGS. 2 and 3 reference numbers 10-16 of the drawings, like numbers indicate like elements throughout the several views of the embodiment of an inhaler device as illustrated, presented here as a non-limiting examples.

FIG. 2 illustrates a side view of an embodiment of the invention, where 10 designates the inhaler body, 11 designates the mouthpiece, 12 designates the air inlet and 13 designates the internal desiccant material on the inside of the air inlet.

FIG. 3 illustrates in a similar fashion a side view of another embodiment, here also indicating the slide 14 for a loaded dose container 15 and a closing off valve 16 for the air inlet.

In a further preferred embodiment of the present invention, a selected, sealed dose container, optionally comprising more doses than one, is inserted in a DPI as described in U.S. Pat. No. 6,422,236, which document is incorporated herein by reference. A container is opened and the enclosed, metered dose is immediately sucked up by an applied, user-initiated suction during a single inhalation effort, whereby the delivered fine particle dose by weight amounts to at least 30%, preferably at least 50% and most preferably at least 70% or more of the active pharmaceutical ingredient(s) of the metered dose, even in specified humid ambient conditions. The present invention is advantageously applied to such a sealed container and inhaler arrangement, whereby retention of powder in the container is minimized and not exceeding 20%, preferably not exceeding 10% and most preferably not exceeding 5% of the active pharmaceutical ingredient(s) of the metered dose by mass, even in specified humid ambient conditions.

An inhaler providing delivery of a dose during the course of a single inhalation from a sealed dose container constitutes an inhaler, which would benefit from the present invention, for improving the delivery of a moisture sensitive dry powder medicament formulation. An Air-razor method as described in U.S. Pat. No. 6,840,239 and an Air-razor device as described in U.S. Pat. No. 6,892,727, which documents are incorporated herein by reference, are preferably applied in the inhaler to efficiently and gradually aerosolize the dose when delivered to the user.

The present invention is suitable for many kinds of dry powder drug formulations and powders produced by different methods and processes, e.g. spray-drying, freeze-drying, super critical crystallisation, jet milling and other types of micronization. Formulations may contain one or more pure active pharmacologic ingredients (API's) or a formulation may comprise pure API's and excipients, in mixtures of powders or ingredients integrated into particles.

Areas of therapy where the present invention is advantageously applied include asthma, COPD and pain. Other examples of therapy areas, not limiting the scope of the invention, include non-exclusively:
Metabolic disorders
Disorders of the alimentary tract or the digestive system
Disorders of the cardiovascular system
Disorders of the endocrine system
Disorders of the respiratory system
Genital or sexual disorders
Disorders of the muscular or neuromuscular system
Disorders of the nervous system
Psychosomatic disorders
Anti-infectives
Allergic disorders
Protective or antinoxious agents

Non-limiting examples of suitable medicaments in dry powder form, which are eminently suitable for delivery of dosages by the present invention—whether in pure or diluted formulations, in single preparations or in combination with other active substances—are insulin, sumatriptan, fluticasone, formoterol and tiotropium to name but a few.

As used herein, the phrases “selected from the group consisting of,” “chosen from,” and the like include mixtures of the specified materials.

All references, patents, applications, tests, standards, documents, publications, brochures, texts, articles, instructions, etc. mentioned herein are incorporated herein by reference. Where a numerical limit or range is stated, the endpoints are included. Also, all values and sub-ranges within a numerical limit or range are specifically included as if explicitly written out.

What has been said in the foregoing is by example only and many variations to the disclosed embodiments may be obvious to a person of ordinary skill in the art, without departing from the spirit and scope of the invention as defined in the appended claims.

We claim:

1. A dry powder inhaler device, comprising
an air inlet, a mouthpiece, and at least one flow channel connecting the mouthpiece with the inlet air, said device adapted to hold and deliver at least one dry powder medicament dose;
the inhaler device further comprising a desiccant material arranged in at least one of said flow channel(s) located downstream of the air inlet such that when at least one dry powder medicament dose is present in said device and suction is applied to the mouthpiece, at least part of an induced stream of air passes through the desiccant, is at least partly dried, and then releases at least a part of the dose and carries entrained powder out of the mouthpiece.

2. The inhaler device according to claim 1, wherein
the desiccant material is filled in a cartridge removably adapted for insertion in the at least one flow channel of the inhaler device.

3. The inhaler device according to claim 1, wherein
the desiccant material is filled in, or made part of, the dose container, in a manner such that the desiccant becomes arranged in the at least one flow channel, and
the desiccant of the container replaces partly or completely the desiccant material in the at least one flow channel of the inhaler device.

4. The inhaler device according to claim 1, wherein
an air-razor method and device are applied in the inhaler device to release the medication dose gradually.

5. The inhaler device according to claim 1, wherein
the device is an active inhaler device, using pressurized ambient air to release the dose, optionally using a spacer for receiving the aerosolized dose prior to inhalation, and
the pressure chamber in the device receives and stores under pressure the partly dried ambient air having first passed through the desiccant, such that the stored, partly dried air is used to aerosolize the dose just prior to an inhalation effort.

6. The inhaler device according to claim 1, wherein
the desiccant mass is appropriate to last for the time the inhaler device is in use according to a set specification for the inhaler and the doses.

7. The inhaler device according to claim 6, wherein
the desiccant mass is chosen to be in a range from 2 to 50 g.

8. The inhaler device according to claim 6, wherein
the desiccant mass is chosen to last for a number of doses between 200 and 500 off.

9. The inhaler device according to claim 1, wherein
the inhaler further comprises means for closing the device, when not needed by the user, such that the desiccant is not subjected to ambient air and humidity between releases of doses.

10. The inhaler device according to claim 1, wherein
the inhaler further comprises alternative routes for the air-stream inside the device when the dose has been fully aerosolized, such that when the dose has been delivered the air-stream bypasses the desiccant, whereby the desiccant is not subjected to moist air when not needed.

11. The inhaler device according to claim 1, wherein
the desiccant is adapted to adsorb moisture only when the relative humidity of the air streaming through the desiccant is above a selected minimum value to be defined in a range from 40% to 75%.

12. The inhaler device according to claim 1, wherein
the desiccant is selected from a group of materials comprising silica gels (SiO₂), activated alumina (Al₂O₃), molecular sieves and clays.

13. A method, comprising improving a delivery performance of a dry powder inhaler device in delivering a dry powder dose of a moisture sensitive dry powder medicament and arranging a desiccant material in at least one flow channel for ambient air flowing into the inhaler device, whereby the relative humidity of the air releasing the dose is significantly reduced.

14. The method according to claim 13, comprising the further step of
filling the desiccant material into a cartridge removably adapted for insertion in the at least one flow channel of the inhaler device.

15. The method according to claim 13, comprising the further steps of
filling the desiccant material in, or making it part of, the dose container, in a manner such that the desiccant becomes arranged in the at least one flow channel, and
the desiccant of the container replaces partly or completely the desiccant material in the at least one flow channel of the inhaler device.

16. The method according to claim 13, comprising the further step of
applying an Air-razor method and device in the inhaler device to release the medication dose gradually.

17. The method according to claim 13, comprising the further steps of
using an active inhaler device, using pressurized ambient air to release the dose, optionally using a spacer for receiving the aerosolized dose prior to inhalation, and
the pressure chamber in the device receives and stores under pressure the partly dried ambient air having first passed through the desiccant, such that the stored, partly dried air is used to aerosolize the dose just prior to an inhalation effort.

18. The method according to claim 13, comprising the further step of
adapting the desiccant mass to last for the time the inhaler device is in use according to a set specification for the inhaler and the doses.

19. The method according to claim 18, comprising the further step of
choosing the desiccant mass to be in a range from 2 to 50 g.

20. The method according to claim 18, comprising the further step of
the desiccant mass is chosen to last for a number of doses between 200 and 500 off.

21. The method according to claim 13, comprising the further step of
closing the inhaler when not needed by the user, such that the desiccant is not subjected to ambient air and humidity between releases of doses.

22. The method according to claim 13, comprising the further step of
arranging alternative routes for the air-stream inside the device when the dose has been fully aerosolized, such that when the dose has been delivered the air-stream bypasses the desiccant, whereby the desiccant is not subjected to moist air when not needed.

23. The method according to claim 13, comprising the further step of
the desiccant is adapted to adsorb moisture only when the relative humidity of the air streaming through the desiccant is above a selected minimum value to be defined in a range from 40% to 75%.

24. The method according to claim 13, comprising the further step of
selecting the desiccant from a group of materials comprising silica gels (SiO₂), activated alumina (Al₂O₃), molecular sieves and clays.

25. A method comprising
selecting by a user a pre-metered, dry powder medicament dose or metering such a dose from an internal powder storage and placing said dose in a position to be inhaled from the inhaler device;
arranging a desiccant in at least one flow channel for ambient air into the device, whereby the air passing through the desiccant is dried, at least partly, before the air is directed towards the powder particles of the dose, and
protecting the dose upon inhalation by the at least partly dried air, such that the intended performance of the inhaler device is maintained even in high humidity ambient conditions.
